(19) United States
(12) Patent Application Publication
(10) Pub. No.: US 2025/0077967 A1
(43) Pub. Date: Mar. 6, 2025

(54) SYSTEMS AND METHODS FOR ACCELERATE SPEED TO MARKET FOR IMPROVED PLANT-BASED PRODUCTS

(71) Applicant: Benson Hill, Inc., St. Louis, MO (US)

(72) Inventors: Jason Bull, St. Louis, MO (US); Nick Darby, St. Louis, MO (US); Dylan Kesler, St. Louis, MO (US); Craig Rolling, St. Louis, MO (US); Paul Skroch, St. Louis, MO (US)

(21) Appl. No.: 18/725,368

(22) PCT Filed: Dec. 29, 2022
(86) PCT No.: PCT/US22/54252

§ 371 Date: Jun. 28, 2024

Related U.S. Application Data
(60) Provisional application No. 63/295,295, filed on Dec. 30, 2021.
(60) Provisional application No. 63/295,664, filed on Dec. 31, 2021.
(60) Provisional application No. 63/295,798, filed on Dec. 31, 2021.
(60) Provisional application No. 63/295,823, filed on Dec. 31, 2021.
(60) Provisional application No. 63/295,826, filed on Dec. 31, 2021.
(60) Provisional application No. 63/326,745, filed on Apr. 1, 2022.
(60) Provisional application No. 63/295,822, filed on Dec. 31, 2021.

(51) Int. Cl. G06N 20/00; G06F 30/27

(52) U.S. Cl. CPC G06N 20/00; G06F 30/27

(57) ABSTRACT

A computer-based method for training and subsequently applying a machine-learning (ML) model to accelerate development of improved plant-based products comprising: (a) collecting into a database seed data including at least parentage information with genetics; (b) training a first ML model based on seed data collected for each data type for each seed variety; (c) establishing a functional specification for the plant-based product; (d) extracting plant traits needed to meet the functional specification; (e) inputting those plant traits into the trained first ML model to generate a predictive breeding crosses list ranked on probability that a progeny of a cross will substantially conform to one or more of those plant traits; (f) collecting data from the progeny planted based on the crosses list; and (g) comparing the collected progeny data to corresponding predictions made by the first ML model toward determining next action recommended by the first ML model.

### CROSS REFERENCE TO RELATED APPLICATIONS

This application claims the benefit and priority of U.S. Provisional Patent Application Ser. No. 63/295,664 filed Dec. 31, 2021 entitled “Systems And Methods For Training A Machine-Learning Model And Subsequently Applying That Machine Learning Model To Accelerate Speed To Market For Improved Plant-Based Products,” U.S. Provisional Patent Application Ser. No. 63/295,826 filed Dec. 31, 2021 entitled “Systems And Methods For Predictive Advancement In A Breeding Pipeline,” U.S. Provisional Patent Application Ser. No. 63/295,823 filed Dec. 31, 2021 entitled “Systems And Methods For Automated Predictive Breeding Workflow,” U.S. Provisional Patent Application Ser. No. 63/295,798 filed Dec. 31, 2021 entitled “Systems And Methods For Recommending Gene Editing Strategies,” U.S. Provisional Patent Application Ser. No. 63/326,745 filed Apr. 1, 2022 entitled “Systems And Methods For Accelerate Speed To Market For Improved Plant-Based Products,” the disclosures of which are hereby incorporated by references in their entirety.

### BACKGROUND

Genomics has been used for decades to develop crops for our food system, but most agricultural companies have focused almost exclusively on increasing the yield of a few crops, resulting in commodity ingredients and a food system based on the quantity of calories available. While focus on quantity is important, that focus resulted in lower nutrient density and changed flavors. Minimal diversity in ingredient options also led food manufacturers to add costly water- and energy-intensive processing steps, and additives like sugar and salt to make up for attributes that were muted in crops over time.

However, consumers are now demanding food choices with simpler ingredients that benefit their health and the health of our planet. Food- and diet-related health issues, including obesity and diabetes, are some of the most widespread health issues today and continue to increase. More than 65% of American adults are either overweight or have obesity and, according to the Centers for Disease Control and Prevention, approximately 90% of Americans do not eat the recommended daily amount of fruits and vegetables. Americans spend more on diet-related illnesses than on food itself.

Moreover, the current food system has a substantial environmental impact on the planet. According to an April 2020 report entitled “Agriculture and climate change” prepared by McKinsey & Company, twenty-seven percent of total greenhouse gas emissions (e.g., methane and nitrous oxide) are caused by agriculture, with cattle and dairy cows alone contributing eight gigatons of carbon dioxide equivalent (GtCO2e) emissions in 2019. (Accessed Dec. 22, 2021 at https://www.mckinsey.com/˜/media/mckinsey/industries/agriculture/our %20insights/reducing %20agriculture %20emissions %20through %20improved %20farming %20practices/agriculture-and-climate-change.pdf.)

At the same time, demand for plant-based solutions to feed the world and improve the environment is growing. Consumers are open to changing their eating habits to minimize further harm to the environment. Moreover, people are actively trying to incorporate more plant based foods into their diets, especially protein alternatives found in the meat and dairy grocery store sections. NielsenIQ Sep. 9, 2021 article entitled “Growing demand for plant-based proteins” (Accessed Dec. 22, 2021 at https://nielseniq.com/global/en/insights/analysis/2021/examining-shopper-trends-in-plant-based-proteins-accelerating-growth-across-mainstream-channels/).

The largest commercial source of plant protein today is the soybean plant. Other plant-based protein crops include chickpeas, edamame, lentils, peanuts, and peas.

### Soybeans: Generally

Soybeans are believed to have originated on the Asian Continent (Glycine soja) where it is believed they were also first domesticated in China (Glycine max). Abstract, Hymowitz and Newell, Taxonomy of the genus Glycine, domestication and uses of soybeans. Econ Bot 35, 272-288 (1981). Soybeans are a common field crop with the largest producing countries including the United States, Brazil, Argentina, China, India, Paraguay, and Canada. In the United States in 2020 soybeans were primarily produced in the Western Corn Belt (48.7%), Eastern Corn Belt (32.7%), and the Midsouth (11.9%) with Illinois and Iowa being the largest producing states. Naeve and Miller-Garvin, United States Soybean Quality 2020 Annual Report (Published by the University of Minnesota with the support of the United Soybean Board).

Soybean plants produce seed-bearing pods, each generally having 2-4 seeds. The seeds are harvested and processed either for future planting (i.e., to produce additional soybean plants) or processed into dozens of products (e.g., bean curd, feed for livestock, flour, meal, oil (cooking and industrial)). Soy flours includes flour concentrates and isolates, which are the primary protein products of soy.

Soybean seeds are usually planted in rows in soil. According to the 2012 Illinois Soybean Production Guide, soybeans require 55-60° F. soil temperature, an air temperature of at least 68° F., about 25 inches of water, sufficient nitrogen and five months from germination to harvest.

The radical (or root) is the first structure to emerge from a germinating soybean seed. The hypocotyl is the seedling structure that emerges from the soil surface. As the hypocotyl emerges it forms a crook as it pulls the cotyledons (i.e., the plant's first leaves) from the soil. Then, the cotyledons can unfold and begin the process of photosynthesis. Once the cotyledons have emerged from the soil surface the plant is said to be at the VE stage of vegetative development. The VC (cotyledon) development stage occurs once two unifoliate (or single blade) leaves emerge from opposite sides of the main stem and no longer touch the cotyledons. The V1 (vegetative) development stage occurs once the unifoliate leaves are fully expanded establishing the first node. V2 is defined as the stage wherein a second node (with a trifoliate leaf (i.e. three or four leaflets per leaf)) has formed above the unifoliate node. With the formation of each subsequent node “n” (n=3, 4 . . . ) with fully developed leaves the plant is referred to as being in the Vn development stage. Soybean farmers typically refer to the leaves and stems as the canopy.

The length of time for these vegetative and reproductive stages (discussed below) depends on the plant's maturity group (“MG” (i.e., the length of time from planting to physical maturity), the soil and air temperatures, and day length. Soybeans are short-day plants (i.e., the soybean plant is triggered to flower as the day length decreases below some critical value, which differs among MGs). See, e.g., Purcell, Salmeron and Ashlock, “Chapter 2: Soybean Growth and Development” Arkansas Soybean Production Handbook (University of Arkansas Division of Agricultural Research & Extension, 2014 Update). Soybeans planted in Arkansas tend to be MG3 through MG6. Id. In Illinois, where soybeans may be grown in regions traditionally understood to be in MG2 through MG5, the 2012 Illinois Soybean Production Guide notes that MG 5 to MG 8 soybeans tend to be determinate (i.e., they cease vegetative growth when the main stem terminates in a cluster of mature pods) and MG 0 to MG 4.9 tend to be indeterminate (i.e. they develop leaves and flowers simultaneously after flowering begins).

Each soybean plant can produce a lot of flowers. The flowers are small and hidden underneath the leaves of the plant. The number of flowers produced depends upon the number of nodes on the main stem and branches with flower-bearing nodes. Not all flowers produce pods. For those flowers that do produce pods whether the resulting pod produces a full complement of seeds requires ample nitrogen, sugar, other nutrients, and favorable environmental conditions.

When a soybean plant begins to flower, it is referred to as being in its reproductive (R) growth stage. Soybeans are a normally self-pollinating crop, in fact, they have a perfect flower structure for self-pollination. Still, bees have been known to be attracted to soybean flowers and cross-pollinated plants. Where cross-pollination is desired breeders need to intervene to prevent self-pollination: the pistil of a soybean plant can become mature and the anthers can begin to shed pollen before the soybean flowers even bloom, breeders seeking to cross-pollinate need to be proactive.

Soybean plants have eight reproductive stages: R1 (beginning flowering/bloom (i.e., at least one flower)), R2 (full flowering/bloom (i.e., an open flower at one of the two uppermost nodes)), R3 (beginning pod (i.e., a pod measuring 3/16 inch at one of the four uppermost nodes)), R4 (full pod (i.e., a pod measuring ¾ inch at one of the four uppermost nodes)), R5 (beginning seed (i.e., a seed measuring ⅛ inch long in the pod at one of the four uppermost nodes)), R6 (full seed (i.e., a pod containing a green seed that fills the pod at one of the four uppermost nodes)), R7 (beginning maturity (i.e., one normal pod has reached mature pod color)), and R8 (full maturity (i.e., at least 95% of pods have reach full mature color)).

As the days get shorter and the temperatures get cooler, the leaves on soybean plants begin to turn yellow, they subsequently turn brown, fall off, and expose the matured pods of soybeans. The soybeans are now ready to be harvested using combines. The header on the front of the combine cuts and collects the soybean plants. The combine separates the soybeans from their pods and stems, and collects them into some container.

After harvesting the soybeans are processed. The soybeans are cleaned, heat dried, crushed and then flaked. Thereafter, the flake is further processed. The primary method for further processing is referred to as the extraction or solvent process, as it uses organic solvents (e.g. hexane) to recover the soybean oil and protein from the flake. Aside from its substantial use of solvents, this process consumes significant amounts of energy.

### Soybeans: Seed Varieties, Breeding, and Genetic Modification

Today, there are literally thousands of varieties of soybeans. These soybeans are the result of hundreds of years of selective breeding. Selective breeding is the process of selectively propagating plants with more desired traits (often called “phenotypes”) and eliminating plants with less desired phenotypes. Breeding generations are often designed F1, F2, etc, (wherein the “F” stands for “filial”). It may further involve crossing two plants to produce one or more new varieties.

Plant botanists have understood since the days of Gregor Mendel, that plants may exhibit dominant or recessive phenotypes/traits (e.g., seed shape, flower color, seed coat tint, pod shape, unripe pod color, flower location, and plant height). Through his experiments on pea plants, Mendel further taught that the genotype of a particular phenotype is not necessarily correlated because that phenotype may result from homozygous dominant, heterozygous, or homozygous recessive alleles. Where the phenotype is dominant, it will be exhibited by either of the first two zygosities. Whereas a recessive phenotype can only be exhibited by the third, homozygous recessive example.

Homozygous genotypes breed true from generation to generation, while heterozygous genotypes do not. Thus, after finding a desirable phenotype, plant breeders work to develop homozygosity in the population, and then release the resulting pure line as a new variety. For example, hybrid varieties are the result of crossing two homozygous, but unrelated pure lines of a species. The resulting F1 of the cross are all heterozygous. However, by F2 50% of the plants are either homozygous (dominant or recessive) and by F3 heterozygosity is reduced to 25%. Once a desired trait is found in homozygous plants, commercial quantities are produced by replanting the resulting seeds over several generations.

However, it takes time for each generation of plants to grow from seeds to adult plants and time to cross plants once they've produced reproductive organs. These and other practical constraints of biology are natural obstacles for traditional breeding programs and slow the advancement of potential commercial products through the phases of a traditional commercial product pipeline. Moreover, while the foregoing basic principles of plant genetics are relatively straightforward, the issue of creating a commercially-desirable variety is complicated by the fact that breeders cannot isolate a particular phenotype from other traits that might be present elsewhere in the plant's genome. Even in Mendel's pea experiments where he worked with only seven traits, each having at least two phenotypes (e.g., seed color: green or yellow) existing with the six other traits each of which also having multiple phenotypes the number of potential combinations explodes given all the ways the phenotypes of each trait can combine with the phenotypes of all the other traits. In the case of the soybean (i.e., Glycine max), its genome has approximately 1,100M base-pairs packaged into twenty chromosome pairs. Arumuganathan K, Earle E D. Nuclear DNA content of some important plant species. Plant molecular biology reporter. 1991 August; 9(3):208-18. Thus, there are an infinite number of potential genetic combinations within the soybean genome. As should be readily apparent, due to the sheer size of a plant's genome, the number of traits/phenotypes, and other practical constraints of biology, traditional plant breeding requires significant time to establish new phenotypes in a population.

Plants may also be genetically modified. Genetic engineering allows for the introduction of a new trait or even just better control over an existing trait. In 2002, for instance, the majority of the soybean plants grown in the United States were genetically modified for herbicide-tolerance. Sleper and Shannon, “Role of Public and Private Soybean Breeding Programs in the Development of Soybean Varieties Using Biotechnology,” AgBioForum, 6(1&2): 27-32 (2003). There are two predominant approaches to genetic engineering in plants: the gene gun and the Agrobacterium method.

In the gene gun method, the desired gene is coated onto small metal particles and shot within a vacuum chamber using a short, high-velocity pulse of a high-pressure, inert gas (e.g., Helium) toward plants covered by a fine mesh baffle that catches the small metal particles while allowing the gene to continue into the target cell.

In the Agrobacterium method, the tumor inducing region is removed from transfer DNA and replaced with the desired gene and a marker, which are inserted into the tissue of an organism usually by direct inoculation with a culture of transformed Agrobacterium. An antibiotic medium is subsequently introduced to kill the Agrobacterium and remove the marker. Only tissues expressing the marker will survive and possess the gene of interest. These tissues are then grown using tissue culture techniques until a plant is grown and produces seeds. Neither of these methods are particularly easy. Additionally, DNA sequencing is generally desirable to confirm that the host cell now contains the new gene and where the gene inserted.

Among the more desirable characteristics that have been selectively bred in soybeans are increased yield and increased tolerance to various potential environmental stressors (e.g., insects, drought). Unfortunately, according to the United States Soybean Quality 2020 Annual Report (conducted by Naeve and Miller-Garvin of the University of Minnesota), while soybean yields have significantly increased in the United States over the last thirty years, the amount of protein contained in those soybeans has substantially declined over the same time period.

### Using Machine Learning to Improve Agricultural Ingredients

While protein output in soybeans has been decreasing, the demand for plant-based protein has been growing. So much so that the demand will likely not be fully met using current breeding, genetic engineering, agronomic, and processing technologies. The current commodity food system can take on the order of six to ten years to improve crops with quality attributes, assuming the agricultural industry can even find the genetic synergy to create the right germplasm and then figure out how to best enable the desired breeding.

There are other plants that could benefit from improved characteristics.

Machine learning (and other forms of artificial intelligence) are already being used to improve certain outcomes in agriculture. One key to successful machine learning is identifying the right types of data to gather and then using that data to train the right type of model. Another key may include identifying the wrong, unnecessary, or cumbersome data the inclusion of which is either unhelpful in developing the model or unnecessarily slows down or other makes the training process unnecessarily expensive without sufficient improvement of the model.

### SUMMARY OF THE DISCLOSURE

The present disclosure is directed to systems and methods for training a machine learning model and subsequently applying that machine learning model to accelerate speed to market for improved plant-based products. In soy, these potential improvements may comprise increased protein content, decreased oligosaccharides (e.g., raffinose and stachyose), maintaining and/or even improving crop yield, improved consumer experience (e.g., taste, texture, smell), and combinations of the foregoing.

The present disclosure teaches a method for training a machine-learning model and subsequently applying that machine learning model to accelerate speed to market for an improved plant-based product. The method comprising: (a) collecting into a database, with a processor, seed data including at least labelled parentage information that includes genetics information; (b) training, with the processor, a first machine-learning model based on the data collected for each data type for each of the plurality of seed varieties within the germplasm; (c) establishing, via the processor, a functional specification for the improved plant-based product; (d) extracting, with the processor, one or more plant traits needed to at least meet the functional specification; (e) inputting, via the processor, the one or more plant traits needed to at least meet the functional specification into the trained first machine learning model to generate a first predictive breeding crosses list ranked based on aggregate probability that a progeny of the cross will substantially conform to one or more of the one or more plant traits needed to meet the functional specification; (f) collecting data, by the processor, from the progeny of crosses planted based on the first predictive breeding crosses list; and (g) comparing, by the processor, the collected progeny data to corresponding predictions made by the first machine learning model toward determining next action recommended by the first machine learning model.

As disclosed herein, the method may calculate potential crosses for advancement in the breeding pipeline to obtain progeny having desired characteristics or combinations of characteristics and/or traits such as yield, protein, oil, height, and maturity based on simulated and/or historical data. Moreover, the method may also estimate population parameters (e.g. population usefulness, transgressive segregation ratio, parent mean, protein, yield, oil, maturity, height) based on the simulated population phenotypes and may then test different selection algorithms.

As disclosed herein, the method may implement a machine learning model to select to select progeny for field testing based entirely on genotypic data before collecting any phenotypes on the plant lines. In some embodiments, the machine learning model may be a neural network with historical data based on genomic predictions, maturity rating, and market class. In other embodiments, the first machine learning model could be selected from the group comprising supervised learning models, unsupervised learning models, and combinations thereof and different from the first machine learning model. The model may predict, for example, the likelihood that a progeny will advance to the next phase of the breeding pipeline (e.g. “Phase 2”), if it were tested in the previous phase (e.g., “Phase 1”) based on the historical data of the previous phase (e.g., Phase 1). The model may predict breeding advancement for a progeny without using any field data or observed phenotypes for the target progeny. As disclosed herein, it may be possible to predict progeny success in the breeding pipeline using only genomic predictions, product class, and estimated maturity rating based on the parents.

As disclosed herein, the method may be directed to only selecting a plant progeny line for advancement within a pipeline. Such a method would comprise: (A) using a first machine learning model trained using simulated training data to identify a first set of candidate progeny lines from a plurality of candidate progeny lines to advance to a testing phase; and (B) using a second machine learning model trained using historical data to identify a second set of candidate progeny lines from the first set of candidate progeny lines to advance to a phase subsequent to the testing phase. The method may further comprise selecting a training data set, which may comprise a genomic marker set, to train the first and/or second machine learning model. The method may also comprise using the second machine learning model trained to identify the second set of candidate progeny lines to advance to the phase subsequent to the testing phase comprises generative data indicative of which of the first set of candidate progeny lines to advance to commercial use. The method may additionally comprise receiving information about a population of plants, wherein the first set and the second set are progenies of the population, and using the first machine learning model comprises automatically using the first machine learning model in response to receiving the information about the population of plants; and using the second machine learning model comprises automatically using the second machine learning model in response to the first machine learning model identifying the first set.

As disclosed herein, the method may further include generating a first list of potential gene editing targets based on a probability that editing a particular gene will result in a plant that will substantially conform to one or more of the one or more plant traits needed to at least meet the functional specification.

As further disclosed herein, the method may further comprise: (h) selecting, with the processor, a second machine learning model based on the data type of each data element of the training data selected to train the second machine learning model (“second training data”), the second machine learning model selected from the group comprising supervised learning models, unsupervised learning models, and combinations thereof and different from the first machine learning model; (i) training, with the processor, the second machine learning model using the second training data from the database; (j) inputting, via the processor, the one or more plant traits needed to at least meet the functional specification into the trained second machine learning model to generate a second predictive breeding crosses list ranked based on aggregate probability that a progeny of the cross will substantially conform to one or more of the one or more plant traits needed to meet the functional specification and a second list of potential gene editing targets based on a probability that editing a particular gene will result in a plant that will substantially conform to one or more of the one or more plant traits needed to at least meet the functional specification; (k) collecting data, by the processor, from the progeny of crosses planted based on the second predictive breeding crosses list; and (l) comparing the collected progeny data to corresponding predictions made by the second machine learning model toward determining next action recommended by the second machine learning model.

As additional disclosed herein, the method may still further comprise: (m) mediating between the first machine learning model and the second machine learning model to establish an aggregated predictive breeding crosses list based on the first and second predictive breeding crosses lists; (n) collecting data from the progeny of crosses planted based on the aggregated predictive breeding crosses list; (o) comparing the collected progeny data to corresponding predictions made by both the first and the second machine learning models toward determining next action recommended by the first and second machine learning model; and (p) mediating between the first machine learning model and the second machine learning model to determine the best next action recommendation.

The first machine learning model may be paired with an in silico simulation model.

In some embodiments, the method may also comprise automated processes to consume data, such as historical genomic and phenotypic data, select optimized genomic marker sets, select optimized model training sets, select optimal genomic selection models, and provide breeding advancement recommendations. For example, the method may process historical genomic and phenotypic data of a soybean. The method may be automated to process and run analysis on the historical data to get summarized phenotypes of all soybean traits. The method may be automated to then use custom markers for obtaining genomic data from the soybean. The method may be automated to then process and link phenotypes with genotypes as well as germplasm metadata information. The method may be automated to determine the best training model based on genomic distance, selecting the best training model for one or more given soybean trait, training the model for one or more soybean traits, and calculating predictions for phenotypes for a germplasm.

The disclosure further teaches various systems that implement the various methods described herein.

These and other aspects of the disclosure will be further explained below.

### DRAWINGS

The Detailed Description is described with reference to the accompanying figures. The use of the same reference numbers in different instances in the description and the figures may indicate similar or identical items.

FIG. 1 is a diagram of a system and associated methods for accelerating the speed to market for improved plant-based products.

FIG. 1A is a diagram of plant-based production development program (150) shown in FIG. 1.

FIG. 1B is a diagram illustrating the types of data gathered and maintained by the system for each seed associated with the system.

FIG. 1C is an illustration of the basic concept behind the various models used in system 100.

FIG. 1D is a diagram showing the probabilities determined for a particular seed object under a particular set of circumstances.

FIG. 2 is a diagram of features that may be used to train one embodiment of the predictive crossing, predictive recombination, predictive advancement, and predictive deployment models used in the plant-based production development program, which may include one or more types of machine learning models depending upon the type of feature data used.

FIG. 3 is a diagram illustrating the process of potential changes to one or more of the machine-learning models based on live data collection.

FIG. 4 is a block diagram illustration one potential system within which one or more of the inventive concepts disclosed in the present specification may be implemented.

### DETAILED DESCRIPTION

The present invention now will be described more fully hereinafter with reference to the accompanying drawings, which form a part hereof, and which show, by way of illustration, specific exemplary embodiments by which the invention may be practiced. This invention may, however, be embodied in many different forms and should not be construed as limited to the embodiments set forth herein; rather, these embodiments are provided so that this disclosure will be thorough and complete, and will fully convey the scope of the invention to those skilled in the art. Among other things, the present invention may be embodied as methods or devices. The following detailed description is, therefore, not to be taken in a limiting sense.

In the following detailed description of embodiments of the inventive concepts, numerous specific details are set forth in order to provide a more thorough understanding of the inventive concepts. However, it will be apparent to one of ordinary skill in the art that the inventive concepts within the disclosure may be practiced without these specific details. In other instances, certain well-known features may not be described in detail to avoid unnecessarily complicating the instant disclosure.

As used herein, the terms “comprises,” “comprising,” “includes,” “including,” “has,” “having,” or any other variation thereof, are intended to cover a non-exclusive inclusion. For example, a process, method, article, or apparatus that comprises a list of elements is not necessarily limited to only those elements but may include other elements not expressly listed or inherently present therein.

Unless expressly stated to the contrary, “or” refers to an inclusive or and not to an exclusive or. For example, a condition A or B is satisfied by anyone of the following: A is true (or present) and B is false (or not present), A is false (or not present) and B is true (or present), and both A and B are true (or present).

The term “and combinations thereof” as used herein refers to all permutations or combinations of the listed items preceding the term. For example, “A, B, C, and combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. A person of ordinary skill in the art will understand that typically there is no limit on the number of items or terms in any combination, unless otherwise apparent from the context.

In addition, use of the “a” or “an” are employed to describe elements and components of the embodiments herein. This is done merely for convenience and to give a general sense of the inventive concepts. This description should be read to include one or at least one and the singular also includes the plural unless it is obvious that it is meant otherwise.

The use of the terms “at least one” and “one or more” will be understood to include one as well as any quantity more than one, including, but not limited to, each of, 2, 3, 4, 5, 10, 15, 20, 30, 40, 50, 100, and all integers and fractions, if applicable, therebetween. The terms “at least one” and “one or more” may extend up to 100 or 1000 or more, depending on the term to which it is attached; in addition, the quantities of 100/1000 are not to be considered limiting, as higher limits may also produce satisfactory results.

Further, as used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

As used herein qualifiers such as “about,” “approximately,” and “substantially” are intended to signify that the item being qualified is not limited to the exact value specified, but includes some slight variations or deviations therefrom, caused by measuring error, manufacturing tolerances, stress exerted on various parts, wear and tear, and combinations thereof, for example.

As used herein, “components” may be analog or digital components that perform one or more functions. The term “component” may include hardware, such as a processor (e.g., microprocessor), a combination of hardware and software, and/or the like. Software may include one or more computer executable instructions that when executed by one or more components cause the component to perform a specified function. It should be understood that any and all algorithms described herein may be stored on one or more non-transitory memory. Exemplary non-transitory memory may include random access memory, read only memory, flash memory, and/or the like. Such non-transitory memory may be electrically based, optically based, and/or the like.

As used herein, a “mutation” is any change in a nucleic acid sequence. Nonlimiting examples comprise insertions, deletions, duplications, substitutions, inversions, and translocations of any nucleic acid sequence, regardless of how the mutation is brought about and regardless of how or whether the mutation alters the functions or interactions of the nucleic acid. For example and without limitation, a mutation may produce altered enzymatic activity of a ribozyme, altered base pairing between nucleic acids (e.g. RNA interference interactions, DNA-RNA binding, etc.), altered mRNA folding stability, and/or how a nucleic acid interacts with polypeptides (e.g. DNA-transcription factor interactions, RNA-ribosome interactions, gRNA-endonuclease reactions, etc.). A mutation might result in the production of proteins with altered amino acid sequences (e.g. missense mutations, nonsense mutations, frameshift mutations, etc.) and/or the production of proteins with the same amino acid sequence (e.g. silent mutations). Certain synonymous mutations may create no observed change in the plant while others that encode for an identical protein sequence nevertheless result in an altered plant phenotype (e.g. due to codon usage bias, altered secondary protein structures, etc.). Mutations may occur within coding regions (e.g., open reading frames) or outside of coding regions (e.g., within promoters, terminators, untranslated elements, or enhancers), and may affect, for example and without limitation, gene expression levels, gene expression profiles, protein sequences, and/or sequences encoding RNA elements such as tRNAs, ribozymes, ribosome components, and microRNAs.

Methods disclosed herein are not limited to mutations made in the genomic DNA of the plant nucleus. For example, in certain embodiments a mutation is created in the genomic DNA of an organelle (e.g. a plastid and/or a mitochondrion). In certain embodiments, a mutation is created in extrachromosomal nucleic acids (including RNA) of the plant, cell, or organelle of a plant. Nonlimiting examples include creating mutations in supernumerary chromosomes (e.g. B chromosomes), plasmids, and/or vector constructs used to deliver nucleic acids to a plant. It is anticipated that new nucleic acid forms will be developed and yet fall within the scope of the claimed invention when used with the teachings described herein.

Methods disclosed herein are not limited to certain techniques of mutagenesis. Any method of creating a change in a nucleic acid of a plant can be used in conjunction with the disclosed invention, including the use of chemical mutagens (e.g. methanesulfonate, sodium azide, aminopurine, etc.), genome/gene editing techniques (e.g. CRISPR-like technologies, TALENs, zinc finger nucleases, and meganucleases), ionizing radiation (e.g. ultraviolet and/or gamma rays) temperature alterations, long-term seed storage, tissue culture conditions, targeting induced local lesions in a genome, sequence-targeted and/or random recombinases, etc. It is anticipated that new methods of creating a mutation in a nucleic acid of a plant will be developed and yet fall within the scope of the claimed invention when used with the teachings described herein.

Similarly, the embodiments disclosed herein are not limited to certain methods of introducing nucleic acids into a plant and are not limited to certain forms or structures that the introduced nucleic acids take. Any method of transforming a cell of a plant described herein with nucleic acids are also incorporated into the teachings of this innovation, and one of ordinary skill in the art will realize that the use of particle bombardment (e.g. using a gene-gun), Agrobacterium infection and/or infection by other bacterial species capable of transferring DNA into plants (e.g., Ochrobactrum sp., Ensifer sp., Rhizobium sp.), viral infection, and other techniques can be used to deliver nucleic acid sequences into a plant described herein. Methods disclosed herein are not limited to any size of nucleic acid sequences that are introduced, and thus one could introduce a nucleic acid comprising a single nucleotide (e.g. an insertion) into a nucleic acid of the plant and still be within the teachings described herein. Nucleic acids introduced in substantially any useful form, for example, on supernumerary chromosomes (e.g. B chromosomes), plasmids, vector constructs, additional genomic chromosomes (e.g. substitution lines), and other forms is also anticipated. It is envisioned that new methods of introducing nucleic acids into plants and new forms or structures of nucleic acids will be discovered and yet fall within the scope of the claimed invention when used with the teachings described herein.

Methods disclosed herein include conferring desired traits to plants, for example, by mutating sequences of a plant, introducing nucleic acids into plants, using plant breeding techniques and various crossing schemes, etc. These methods are not limited as to certain mechanisms of how the plant exhibits and/or expresses the desired trait. In certain nonlimiting embodiments, the trait is conferred to the plant by introducing a nucleotide sequence (e.g. using plant transformation methods) that encodes production of a certain protein by the plant. In certain nonlimiting embodiments, the desired trait is conferred to a plant by causing a null mutation in the plant's genome (e.g. when the desired trait is reduced expression or no expression of a certain trait). In certain nonlimiting embodiments, the desired trait is conferred to a plant by crossing two plants to create offspring that express the desired trait. It is expected that users of these teachings will employ a broad range of techniques and mechanisms known to bring about the expression of a desired trait in a plant. Thus, as used herein, conferring a desired trait to a plant is meant to include any process that causes a plant to exhibit a desired trait, regardless of the specific techniques employed.

As used herein, “fertilization” and/or “crossing” broadly includes bringing the genomes of gametes together to form zygotes but also broadly may include pollination, syngamy, fecundation and other processes related to sexual reproduction. Typically, a cross and/or fertilization occurs after pollen is transferred from one flower to another, but those of ordinary skill in the art will understand that plant breeders can leverage their understanding of fertilization and the overlapping steps of crossing, pollination, syngamy, and fecundation to circumvent certain steps of the plant life cycle and yet achieve equivalent outcomes, for example, a plant or cell of a soybean cultivar described herein. In certain embodiments, a user of this innovation can generate a plant of the claimed invention by removing a genome from its host gamete cell before syngamy and inserting it into the nucleus of another cell. While this variation avoids the unnecessary steps of pollination and syngamy and produces a cell that may not satisfy certain definitions of a zygote, the process falls within the definition of fertilization and/or crossing as used herein when performed in conjunction with these teachings. In certain embodiments, the gametes are not different cell types (i.e. egg vs. sperm), but rather the same type and techniques are used to effect the combination of their genomes into a regenerable cell. Other embodiments of fertilization and/or crossing include circumstances where the gametes originate from the same parent plant, i.e. a “self” or “self-fertilization”. While selfing a plant does not require the transfer pollen from one plant to another, those of skill in the art will recognize that it nevertheless serves as an example of a cross, just as it serves as a type of fertilization. Thus, methods and compositions taught herein are not limited to certain techniques or steps that must be performed to create a plant or an offspring plant of the claimed invention, but rather include broadly any method that is substantially the same and/or results in compositions of the claimed invention.

A “plant” refers to a whole plant, any part thereof, or a cell or tissue culture derived from a plant, comprising any of: whole plants, plant components or organs (e.g., leaves, stems, roots, etc.), plant tissues, seeds, plant cells, protoplasts and/or progeny of the same. A plant cell is a biological cell of a plant, taken from a plant or derived through culture of a cell taken from a plant.

A “population” means a set comprising any number, including one, of individuals, objects, or data from which samples are taken for evaluation, e.g. estimating QTL effects and/or disease tolerance. Most commonly, the terms relate to a breeding population of plants from which members are selected and crossed to produce progeny in a breeding program. A “population of plants” can include the progeny of a single breeding cross or a plurality of breeding crosses and can be either actual plants or plant derived material, or in silico representations of plants. The member of a population need not be identical to the population members selected for use in subsequent cycles of analyses nor does it need to be identical to those population members ultimately selected to obtain a final progeny of plants. Often, a “plant population” is derived from a single biparental cross but can also derive from two or more crosses between the same or different parents. Although a population of plants can comprise any number of individuals, those of skill in the art will recognize that plant breeders commonly use population sizes ranging from one or two hundred individuals to several thousand, and that the highest performing 5-20% of a population is what is commonly selected to be used in subsequent crosses in order to improve the performance of subsequent generations of the population in a plant breeding program.

“Crop performance” is used synonymously with “plant performance” and refers to of how well a plant grows under a set of environmental conditions and cultivation practices. Crop performance can be measured by any metric a user associates with a crop's productivity (e.g. yield), appearance and/or robustness (e.g. color, morphology, height, biomass, maturation rate), product quality (e.g. fiber lint percent, fiber quality, seed protein content, seed carbohydrate content, etc.), cost of goods sold (e.g. the cost of creating a seed, plant, or plant product in a commercial, research, or industrial setting) and/or a plant's tolerance to disease (e.g. a response associated with deliberate or spontaneous infection by a pathogen) and/or environmental stress (e.g. drought, flooding, low nitrogen or other soil nutrients, wind, hail, temperature, day length, etc.). Crop performance can also be measured by determining a crop's commercial value and/or by determining the likelihood that a particular inbred, hybrid, or variety will become a commercial product, and/or by determining the likelihood that the offspring of an inbred, hybrid, or variety will become a commercial product. Crop performance can be a quantity (e.g. the volume or weight of seed or other plant product measured in liters or grams) or some other metric assigned to some aspect of a plant that can be represented on a scale (e.g. assigning a 1-10 value to a plant based on its disease tolerance).

A “microbe” will be understood to be a microorganism, i.e. a microscopic organism, which can be single celled or multicellular. Microorganisms are very diverse and include all the bacteria, archaea, protozoa, fungi, and algae, especially cells of plant pathogens and/or plant symbionts. Certain animals are also considered microbes, e.g. rotifers. In various embodiments, a microbe can be any of several different microscopic stages of a plant or animal. Microbes also include viruses, viroids, and prions, especially those which are pathogens or symbionts to crop plants.

A “fungus” includes any cell or tissue derived from a fungus, for example whole fungus, fungus components, organs, spores, hyphae, mycelium, and/or progeny of the same. A fungus cell is a biological cell of a fungus, taken from a fungus or derived through culture of a cell taken from a fungus.

A “pest” is any organism that can affect the performance of a plant in an undesirable way. Common pests include microbes, animals (e.g. insects and other herbivores), and/or plants (e.g. weeds). Thus, a “pesticide” is any substance that reduces the survivability and/or reproduction of a pest, e.g. fungicides, bactericides, insecticides, herbicides, and other toxins.

“Tolerance” or improved tolerance in a plant to disease conditions (e.g. growing in the presence of a pest) will be understood to mean an indication that the plant is less affected by the presence of pests and/or disease conditions with respect to yield, survivability and/or other relevant agronomic measures, compared to a less tolerant, more “susceptible” plant. Tolerance is a relative term, indicating that a “tolerant” plant survives and/or performs better in the presence of pests and/or disease conditions compared to other (less tolerant) plants (e.g., a different soybean cultivar) grown in similar circumstances. As used in the art, tolerance is sometimes used interchangeably with “resistance”, although resistance is sometimes used to indicate that a plant appears maximally tolerant to, or unaffected by, the presence of disease conditions. Plant breeders of ordinary skill in the art will appreciate that plant tolerance levels vary widely, often representing a spectrum of more-tolerant or less-tolerant phenotypes, and are thus trained to determine the relative tolerance of different plants, plant lines or plant families and recognize the phenotypic gradations of tolerance.

A plant, or its environment, can be contacted with a wide variety of “agriculture treatment agents.” As used herein, an “agriculture treatment agent”, or “treatment agent”, or “agent” can refer to any exogenously provided compound that can be brought into contact with a plant tissue (e.g. a seed) or its environment that affects a plant's growth, development and/or performance, including agents that affect other organisms in the plant's environment when those effects subsequently alter a plant's performance, growth, and/or development (e.g. an insecticide that kills plant pathogens in the plant's environment, thereby improving the ability of the plant to tolerate the insect's presence). Agriculture treatment agents also include abroad range of chemicals and/or biological substances that are applied to seeds, in which case they are commonly referred to as “seed treatments” and/or seed dressings. Seed treatments are commonly applied as either a dry formulation or a wet slurry or liquid formulation prior to planting and, as used herein, generally include any agriculture treatment agent including growth regulators, micronutrients, nitrogen-fixing microbes, and/or inoculants. Agriculture treatment agents include pesticides (e.g. fungicides, insecticides, bactericides, etc.) hormones (abscisic acids, auxins, cytokinins, gibberellins, etc.) herbicides (e.g. glyphosate, atrazine, 2,4-D, dicamba, etc.), nutrients (e.g. a plant fertilizer), and/or a broad range of biological agents, for example a seed treatment inoculant comprising a microbe that improves crop performance, e.g. by promoting germination and/or root development. In certain embodiments, the agriculture treatment agent acts extracellularly within the plant tissue, such as interacting with receptors on the outer cell surface. In some embodiments, the agriculture treatment agent enters cells within the plant tissue. In certain embodiments, the agriculture treatment agent remains on the surface of the plant and/or the soil near the plant. In certain embodiments, the agriculture treatment agent is contained within a liquid. Such liquids include, but are not limited to, solutions, suspensions, emulsions, and colloidal dispersions. In some embodiments, liquids described herein will be of an aqueous nature. However, in various embodiments, such aqueous liquids that comprise water can also comprise water insoluble components, can comprise an insoluble component that is made soluble in water by addition of a surfactant, or can comprise any combination of soluble components and surfactants. In certain embodiments, the application of the agriculture treatment agent is controlled by encapsulating the agent within a coating, or capsule (e.g. microencapsulation). In certain embodiments, the agriculture treatment agent comprises a nanoparticle and/or the application of the agriculture treatment agent comprises the use of nanotechnology.

In certain embodiments, plants disclosed herein can be modified to exhibit at least one “desired trait”, and/or combinations thereof. The disclosed innovations are not limited to any set of traits that can be considered desirable, but nonlimiting examples include male sterility, herbicide tolerance, pest tolerance, disease tolerance, modified fatty acid metabolism, modified carbohydrate metabolism, modified seed yield, modified seed oil, modified seed protein, modified lodging resistance, modified shattering, modified iron-deficiency chlorosis, modified water use efficiency, and/or combinations thereof. Desired traits can also include traits that are deleterious to plant performance, for example, when a researcher desires that a plant exhibits such a trait in order to study its effects on plant performance.

In certain embodiments, a user can combine the teachings herein with high-density molecular marker profiles spanning substantially the entire soybean genome to estimate the value of selecting certain candidates in a breeding program in a process commonly known as “genomic selection”.

The term “machine learning” generally refers to computer algorithms that may learn from pre-existing data and then make predictions about new data. Thus, machine-learning tools operate by building a model from example training data, which, for example, can be used to model an environment based on that training data and then make decisions or predictions without explicit instructions.

Different machine-learning tools may be used. Deep learning or deep structured learning is a type of machine learning that can use artificial neural networks (e.g., inspired by biological systems) with representation learning. Representation learning is a set of techniques that allows a system to automatically discover representations needed to detect features in future sets of data.

The learning of features is generally thought to be either supervised or unsupervised, although a hybrid of these approaches is also possible.

In “supervised learning,” a “teacher” presents the computer with the desired outputs given a set of example inputs. This is generally thought to involve classification and regression, which can be accomplished using one or more approaches including, but not limited to, decision trees, ensembles (e.g. Random Forest), nearest neighbors algorithm, linear regression, gBLUP (genomic best linear unbiased prediction), lasso (least absolute shrinkage and selection operator), lasso LARS, Ridge regression, Elastic Net, Naive Bayes, Artificial neural networks (ANN or NN), logistic regression, perceptron, Relevance vector machine (RVM), and Support vector machine (SVM). Generally, the approach to supervised learning used depends on the data set, among other issues involved in this choice is the amount training data available, the dimensionality and heterogeneity of that data, redundancy in that data, the interrelations between data elements, and the amount of noise present in the output.

In “unsupervised learning,” the computer is left to find any naturally occurring patterns within the training data. This can be accomplished by using one or more approaches including, but not limited to, clustering (i.e., automatically grouping the training examples into categories with similar features), anomaly detection, principal component analysis (i.e., automatically identifying features that are most useful for discriminating between different training examples and then discarding the rest), self-organizing feature maps, and latent variable models. Clustering methods include hierarchical clustering, k-means, mixture models (i.e., a probabilistic model that represents the presence of subpopulations within an overall population), DBSCAN (density-based spatial clustering of applications with noise), expectation-maximization, BIRCH, and CURE.

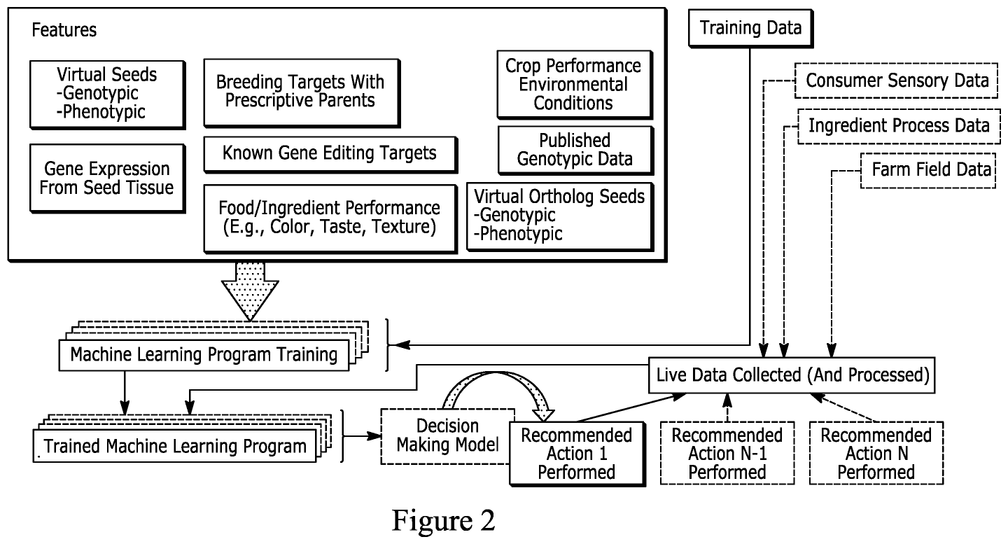

As illustrated in FIG. 2, one or more of the foregoing supervised and unsupervised machine learning approaches may be used by the present system and methods in parallel or seriatim using the same training data or subsets thereof. Where subsets are used the scope of any such subset may be selected for use with the particularly selected training data within that subset with reference to the pluses and minuses of one or more of the particular approaches to machine learning. Where multiple machine learning approaches are used in parallel (i.e., stacked) a decision-making model is preferably introduced to mediate between the probability assessments provided by the multiple machine learning models toward providing a single list of recommended actions (e.g., desirable plant crosses, gene editing targets, crop management techniques).

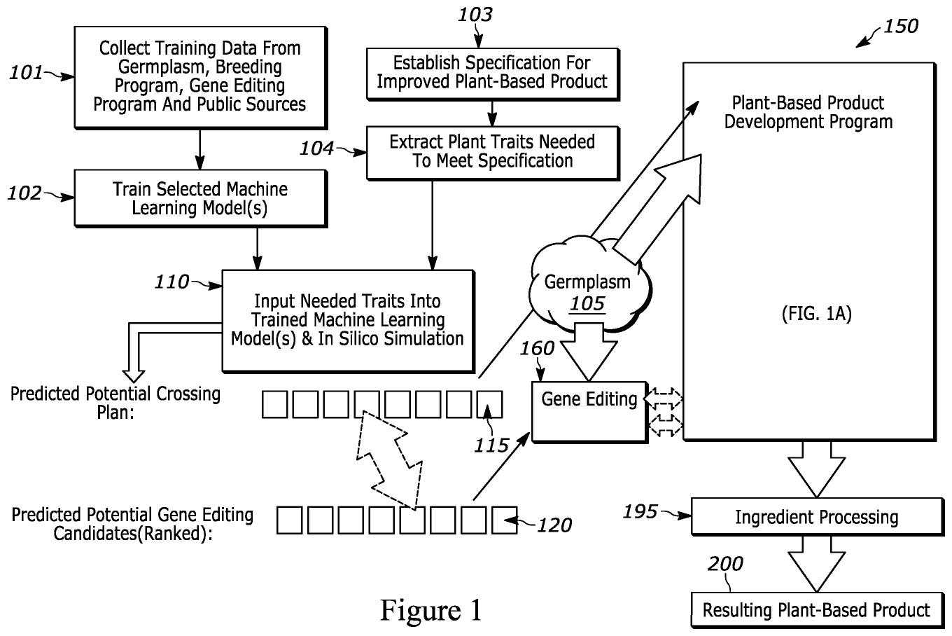

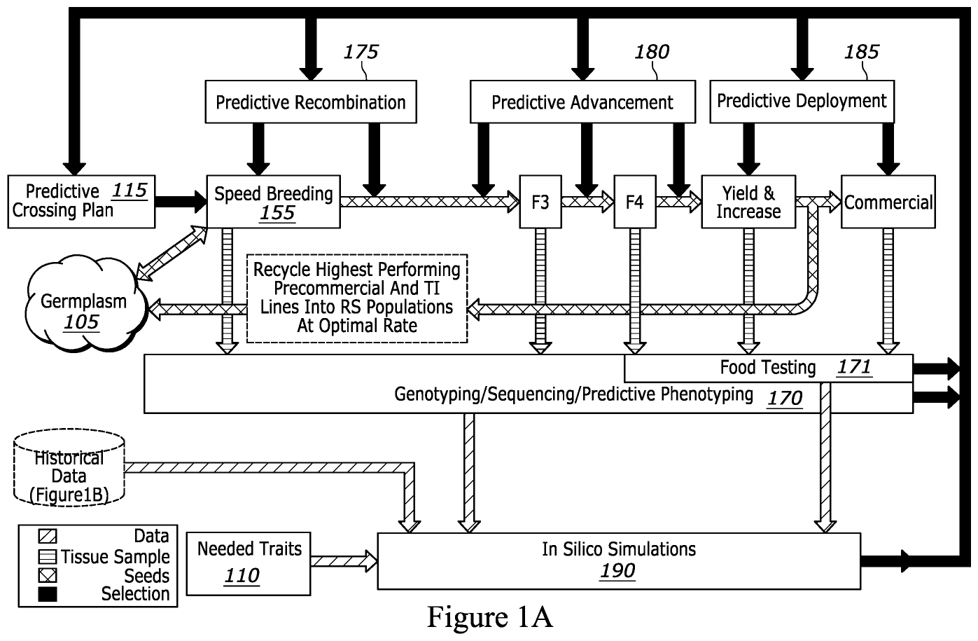

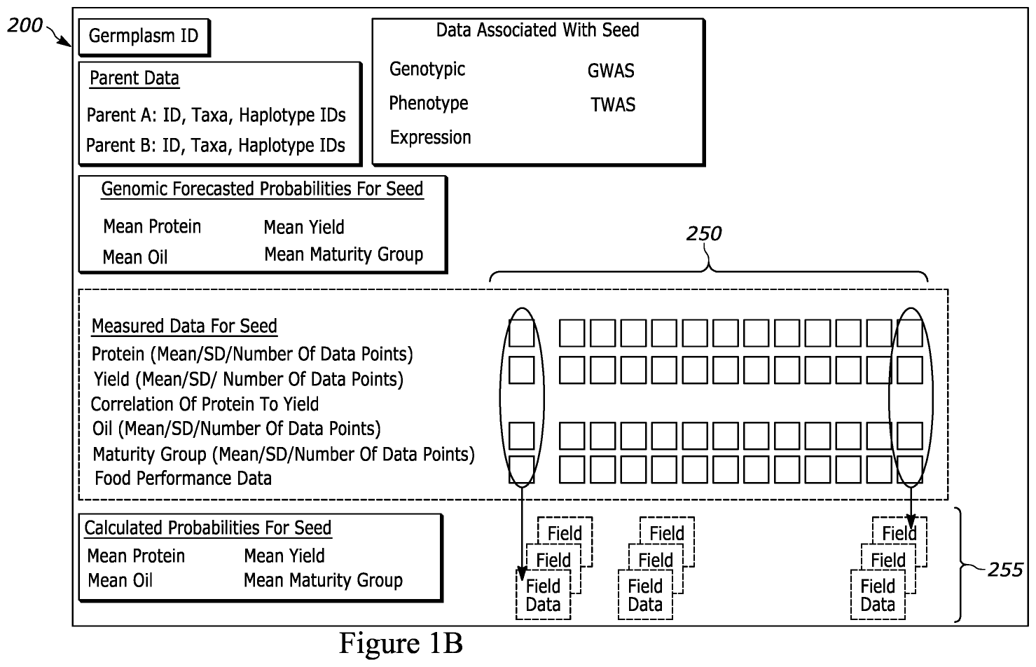

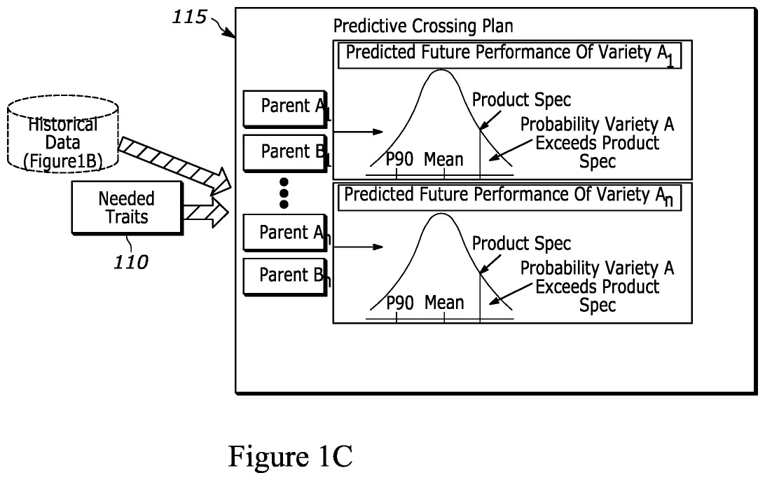

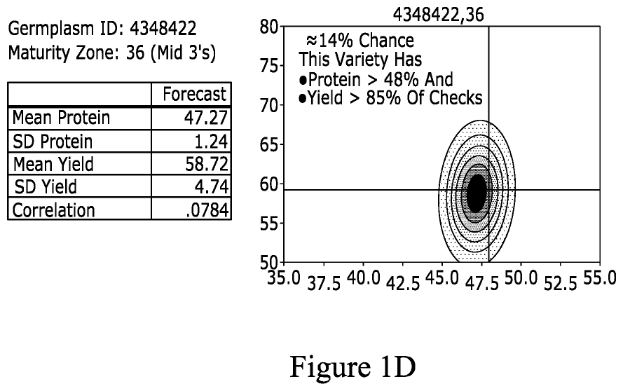

Training machine learning models requires the selection of features and collection of data associated with relevant features in order to appropriately train the machine learning model. As illustrated across FIGS. 1B and 2, the present disclosure identifies various categories of data that the inventors believe may play a substantive role in training useful models. As illustrated in FIG. 1B, the potentially useful data is saved to a seed object (or seed vector) 200 that describes each unique seed contained within the germplasm 105. As illustrated in FIG. 1B, the seed object 200 is preferably identified by one or more of its germplasm ID, its parentage, genotypic, phenotypic, or other genetic data. Seed object 200 may be virtual in the sense that it may contain nothing more than the germplasm ID, parentage and basic genetic data. A “virtual” seed object 200 may also include genomic forecasted probabilities for the seed such as protein content, yield, oil content, and maturity group, all of which may be represented as their mean values and may have an associated standard deviation.

These less fulsome objects/vectors still play a substantive role in simulation and ML decision making. However, as further illustrated by FIGS. 1A, 1B and 2, additional data collected may be added to these seed object/vectors that improve the ability of the models to evaluate and make recommendations with respect to subsequent decisions as to future crosses, recombination, seed advancement, and seed deployment.

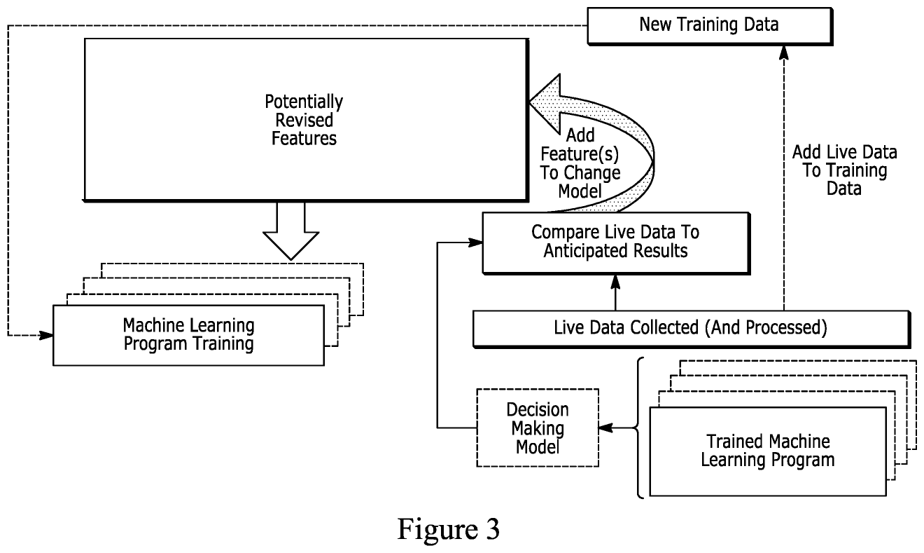

As illustrated across FIGS. 1A, 1, 2 and 3, physical testing data may be collected and may be further processed based on directions from the machine learning system. Processing may be performed on the directly observed physical data (e.g., genotype, phenotype, genetic sequencing (partial or WGS), ingredient processing data, and consumer sensory data) or on one or more derivative data sets (e.g., GWAS or TWAS) based on the observed physical data. The directly observed data may be collected during speed breeding, field testing, and commercialization and/or from the results of such speed breeding, field testing, and commercialization by obtaining tissue samples from the various steps in the process (as illustrated in FIG. 1A). For example, as illustrated in FIG. 1A, tissue samples may be obtained from seeds generated during speed breeding which may be subjected genotyping, sequencing (partial or WGS), and/or predictive phenotyping. In another example illustrated by FIG. 1A, tissue samples taken from seeds resulting from the growth of an F4 generation may be subjected to both food testing protocols as well as genotyping/sequencing/predictive phenotyping, whereas seeds resulting from commercialization may only be subjected to food testing protocols. Thus, during and following one or more events illustrated in FIG. 1A, information is recorded to a seed object 200 associated with a particular seed.

As there will be a variable number of observations across each of the seed object/vector 200 associated with the germplasm 105 as the system 100 continues to operate, the equal variance assumption that underlies linear ML models will not be met. Consequently, operations in the present system may better lend themselves to neural network analysis. Moreover, because neural networks allow the system to capture relationships even where certain outlier values may be “too high” or “too low,” these NNs may provide additional advantages to the system over linear and other models with respect to real-time agricultural decision making.

The data saved to seed object 200 may also include measured data for a seed (really a population of seeds sharing a common pedigree). As illustrated in FIG. 1B, for soybeans this measured seed data 250 may include protein, yield, oil, Maturity Group, and food testing protocol data for each instance that seed is grown. The protein and oil data may be further measured and recorded as to type of protein/oil. Field data 255 for each instance the seed has been grown and observed may also preferably be associated with this collected data. Field data 255 may include location (e.g. latitude/longitude), soil health (e.g., its microbiome and Nitrogen content), climate data (e.g., rain frequency and amount, sunlight length and intensity, air temperature, soil temperature), and applied crop management practices (e.g., planting depth, plant spacing, planting date, irrigation, fertilizer use). As noted above, the field data 255 collected with respect to any particular growing event may not produce instructive data with respect to all of these variables (e.g., the location of an indoor growing event) or even where all of the variables could have been collected, the data may not have been recorded, entered into the dataset, or removed from the dataset for various reasons. In this regard, the models selected for use with the overall dataset contemplate the potential absence of data points from the overall dataset.

As illustrated in FIG. 1B, the record may also include the number of actual data points collected with respect to each separate data type, as well as the mean and standard deviation for that data, and various correlations, such as the correlation of observed protein to observed yield. It should be understood by those of ordinary skill in the art having the present disclosure before them that other correlations may be calculated and included in a seed object data record 200, such as correlations, if any, between protein and oil, protein and maturity group, protein and food testing data, yield and oil, yield and maturity group, yield and food testing data, oil and maturity group, oil and food testing data, and maturity group and food testing data. It may further be possible using the collected data to identify opportunities to use growing data from one or more prior growing season in predicting future performance of the seed. Thus, for example, the probabilities with respect to future protein and yield of a seed, are significantly improved when combining genomic prediction with prior year field data (e.g. use the measured results of Phase 1 field testing to predict Phase 2 results).

The genotypic data may include, but is not limited to, ATAC-Seq, gene annotation, gene expression, genes essential development and maintenance, GO (Gene Ontology) Terms, GWAS (genome wide association study) data, known QTL (quantitative trait locus) data, known eQTL (expression quantitative trait locus), expression data, co-expression data, metabolites data, promoters, RNA-sequencing data (preferably collected at R4 and R5), structural variant (SV) data, transcriptome data, TWAS (transcriptome-wide association study) data, and WGS (whole genome sequencing) data. The matched transcriptome and WGS data may comprise the entirety (or nearly the entirety) of the DNA sequence of an organism's genome. It is further envisioned that a collection of genotypes, some of which may be “haplotypes” at loci that are clustered together on the same chromosome, as well as collections of genotypes from across a single chromosome, and/or collections of genotypes corresponding to loci distributed on different chromosomes may be measured, saved, and used in one or more of the various models operating within the present system.

ATAC-Seq is a technique to assess genome-wide chromatin accessibility. Gene expression links to tissues and times when a particular gene is active allowing for a direct link of gene level changes to phenotypic changes, at scale. Gene Ontology is a representation of detectable observations in genes and relationships between those observations, which allows scientists to publish specific observations about genes opening up literature as a source of training data. GWAS data is a method of studying associations between a genome-wide set of single-nucleotide polymorphisms (SNPs) and a desired phenotypic traits, such as increased protein content. QTL is the location within a genome that correlates with a variation in a quantitative phenotype of the organism.

It is contemplated that some of the data may be collected and fed back into the model continuously. Other data, such as expression data, while it is high value correlative data particularly with respect to protein content in soybeans, is expensive to generate. Assuming a scenario where genotype data for 5,000 samples is approximately $135,000, expression data for just four replicates, 2 tissues would be approximately $9,000,000. In such instances it would be ideal to find a proxy for such data. Continuing with the example of expression data, expression values can be predicted using already collected expression data correlated to other genotypic data. Using predicted expression data allows the system to dramatically increase sample numbers and the power of the machine learning model. In particular, by using the predicted expression for more than 6,300 genes across 1800+ soybean lines along with protein measurements for those same 1800+ soybean lines as training data for a random forest regression machine-learning model, high predictive accuracy has been obtained.

The phenotypic data may include various desirable and undesirable traits associated with a particular plant. For example, with respect to plants used in making plant-based protein product phenotypic data may include the protein content in seeds of the plant (measured both in the field using NIR and in a wet lab), the density of other nutrients in the seeds of the plant, the oil content in seeds of the plant, the oleic acid content in seeds of the plant, the fiber content in the seeds of the plant, the oligosaccharides content (e.g., raffinose and stachyose) in seeds of the plant, the saponins/isoflavones/PUFA content in the seeds of the plant, the content of other off-flavor contributing chemicals (e.g., Hexanal and Hexanol) in the seeds of the plant, the moisture content in seeds of the plant (water holding capacity), plant height, the yield history for the plant, the maturity group (MG) of the plant, and environmental stress resistance of the plant.

It would also be desirable to integrate into the “design” of new plant breeds, food science data for the resulting plant-based end-products, including consumer sensory panel data (e.g., taste (bitterness, richness, saltiness, sourness, umami), texture, firmness, color) and supply chain insights. For instance, water usage, energy usage, and overall cost of producing products based on a particular plant breed design should be fed back into the machine learning model as features to allow for additional improvements in this regard.

At least in the near term, the answer to meaningful substantive improvement of plant-based products may result from the aggregation of smaller improvements in those products. The disclosed systems and methods can consider billions of data points in millions of pipeline configurations to identify the starting parental plant breeding combinations, predict gene targets, and analyze optimal farm management and environmental conditions to guide eventual placement of improved varieties in the field. This result may more easily be attained by assessing the seeds in germplasm 105 using the machine learning techniques disclosed herein alongside in silico simulation and perhaps also gene editing. In fact, using just machine learning and in silico simulation has already facilitated the rapid identification and development of plant-based (soy) products with ultra-high protein (UHP). The ability to get plant-based products to get to market more efficiently and more quickly may be important in effectively responding to evolving consumer preferences and the needs of growers. Such in silico simulation may be enabled in some part, by one or more of the same RNA-sequencing data, structural variant data, whole genome sequences data, phenotype data, and genotype data used to power the machine learning models.

The machine learning model may, among other things, predict potential successful breeding crosses and potential QTLs and/or eQTLs that may provide promising targets to pursue using gene editing and/or breeding techniques based on the knowledge provided to the machine learning program regarding plants and their gene functions. This in turn can provide one or more paths to unlock and/or restore lost or muted genetic variation that is within the natural diversity of the plant and/or knock out genes that result in undesirable traits.

For example, if an improved soy-protein based hamburger is desired, the process begins by setting product specifications, which could include increased protein content, increased water holding capacity, improved flavor, and decreased total oil. The specification could also require that ingredient processing be as energy-efficient as possible to meet growing consumer preferences. In another example, desired specifications for soybean-based white beverage (e.g., soy milk) could include increased protein content, increased solubility, improved flavor, improved color, and a differentiated saturated fat profile. In yet another example, an soybean-based egg replacement, the specification may include increased emulsion/foaming, increased gelation, increased water holding capacity, and decreased total oil. Based on each particular specification, the necessary traits for the ultimately desired commercial soybean for that specification would be established. Then, the work of breeding and gene editing to achieve those desired traits in a commercial soybean plant begins.

As recognized in the prior art, selective breeding to achieve desired traits in plant products takes years. In the method 100 generally illustrated by FIGS. 1 and 1A, the desired traits (e.g., maximized protein content, minimized oligosaccharides, increased water holding capacity), may be assessed against the genetic information and phenotypes of plants within an available germplasm as well as available gene editing targets within that germplasm to predict and potentially rank the most efficient (e.g., quickest, most cost-effective, most environmentally friendly, and combinations thereof) paths that have the highest probabilities of achieving the desired specification. In many cases, some traits will be easier to integrate through gene editing than breeding. In other cases, gene targets believed to result in the desired traits may be yet unknown, too difficult to edit/modify successfully, provide insufficient improvement of the desired trait, or may otherwise prove undesirable. In some cases, a combination of breeding crosses and genetic editing will provide the most efficient path to the desired end product specification. In still other cases, breeding, genetic editing, planting location and crop management techniques will provide the most efficient path with the highest probability of producing an end product that meets (or exceeds) the specification. Thus, the systems and methods disclosed herein employ a diverse array of computation pipeline simulations and machine learning based predictions to execute the most efficient and cost-effective path to novel product development.

In particular, one or more machine learning models are trained (102) using training data collected (101) from one or more of the following: the germplasm 105 (e.g., phenotypic, genotypic), any existing breeding program data (e.g., phenotypic, genomic), any existing gene editing program, as well as publicly available literature and information regarding the plant species underlying the resulting product. A specification is established for the improved plant-based product (103) and the plant traits needed to meet the specification (e.g., protein content, decreased/muted chemical expression) are extracted from the specification (104). The extracted specifications are input into the trained machine learning model(s) and in silico simulation(s) 190. As a result, lists of desirable predicted breeding crosses (preferably by maturity group) (115) and a list of potential gene editing candidates (120) both having been ranked by probabilities determined by the machine learning model(s) may be produced.

At its most basic, the predictive crossing plan 115 (see FIGS. 1 and 1A) is based on the calculated probability of the progeny meeting product thresholds and maximizing genetic value with respect to one or more traits (e.g., protein content). This general concept is illustrated in FIG. 1C with respect to the predicted performance of a single trait for just two of the millions of potential crosses that are actually calculated and assessed by the predictive crossing plan (in addition to the calculations made in the predictive recombination, predictive advancement, and predictive deployment models, as a result of each predicted cross). These calculations may use GEBV (genomic estimate breeding values). FIG. 1D further illustrates the results with the calculation with two traits, protein and yield, for one particular soybean (i.e., the progeny of one particular potential breeding decision) that has been assigned a particular GermplasmID and has a probable maturity group in the middle of Group III (i.e., 36). The results of modeling this particular “seed” determines that the mean protein of the progeny will likely be 47.27% with a standard deviation of 1.24 and a mean yield of 58.72% with a standard deviation of 4.74 and moreover that there is approximately a 14% chance that this variety could have a protein content greater than 48% and a yield “greater than 85% of checks.” In this regard, yield may be measured in terms of the protein recovered per acre as opposed to the more traditional method of measuring yield, i.e. pounds of dry seed obtained per acre.

At a more basic level, the machine learning model may be trained to merely predict advancement of a plant line out of a testing phase. Such a method may using training data to train the machine learning model such that the machine learning model takes as input genotype information about a plurality of candidate plant lines selected for the testing phase without taking as input information about phenotypes of the plurality of candidate plant lines and outputs data indicative of which of the plurality of candidate plant lines should advance out of the testing phase.

As illustrated in FIG. 1A, the plant-based product development program 150 may include speed breeding 155. This speed breeding 155 is likely to be conducted within an indoor facility that provides controlled growing conditions (e.g., temperature, daily photoperiod, humidity) year around without unintentional stressors (e.g., insects, drought). Even though speed breeding and even F3 may be conducted within an indoor facility, it is contemplated that F4 may still be grown outdoors. In speed breeding 155, the daily photoperiod is longer resulting expedited growth in the plants. Speed breeding 155 may include two selection processes: crossing and selfing. Whether any line is advanced, crossed, self-crossed, or back-crossed from one generation to the next may depend upon data gathered from the resulting plants that comprise the line. In this regard, as shown in FIG. 1A, tissue samples may be obtained from the seeds of plants grown in speed breeding 155.

As illustrated in FIG. 1A, tissue samples may be collected from the plants within the speed breeding program 155. These tissue samples may be subjected to a variety of physical tests 170, such as genotyping, sequencing, and predictive phenotyping. In this regard, where a primary specification needed for the resulting plant-based product is increased protein, one type of physical data gathered from the plants may comprise certain NIR data. This NIR data may be correlated to predict protein content in soybeans. The NIR data may be obtained by applying NIR light directly to soybeans, soybean pods, or even soy plants, but most preferably the NIR light is applied directly to the beans. Where other product specifications are sought, other physical testing may be done, as may be appropriate, given the specification and the particular stage in the pipeline (e.g., speed breeding, F3, F4, Yield & Increase, and Commercialization) as illustrated by FIG. 1A.

Some amount of genotype data may also be collected between generations. The collection of this genomic data allows for assessment of the model and better future predictions. Where genomic data of a line significantly deviates from the genomic predictions of the model (especially if that deviation suggests negative future performance), that line may not be further advanced through breeding.

Within the speed breeding program 155, decisions may be made by the predictive recombination model 175 as to selfing/crosses to be performed. Predictive recombination model 175 may receive input from the results of physical testing 170, the output of in silico simulation 190, or both. The results produced by in silico simulation 190 may also be based on the output of one or more component of the physical testing 170, food testing 171, historical genetic or phenotypic data of other seeds in the germplasm 105 (see, e.g., seed object 200 (FIG. 1)). This historical seed data may, itself, be real physical testing observations 170 (which may have been obtained from speed breeding 155 or actually in-field growth), calculated from real physical observations, predicted data, the result of in silico simulation 190, or a combination of one, some or all of the foregoing. The model may adjust based on the source of the data (e.g., real physical observation data versus simulated data versus predicted data).

The predictive recombination model 175 is a machine learning model that directs that particular plants within speed breeding 155 are crossed and/or selfed. The predictive recombination model 175 is preferably trained (and potentially optimized) to achieve a few outcomes: (1) improve overall genetic diversity in the germplasm 105; (2) provide germlines for potential future products; and (3) provide a product focused on meeting the specifications for a particularly desired improved plant-based product. Based on the plants presently growing in speed breeding 155, the predictive recombination model 175 may assess hundreds upon hundreds or even thousands upon thousands of potential breeding options to determine which one(s) of the options have higher probabilities of leading to one or more of the desired outcomes. For example, where predictive recombination model 175 recommends a selfing out of F2, it has assessed that such selfing has a significant probability of meeting the desired product specification in the future.

Some amount of genotype data may also be collected between generations. The collection of this genomic data allows for assessment of the model and better future predictions. Where genomic data of a line significantly deviates from the genomic predictions of the model (especially if that deviation suggests negative future performance), that line may not be further advanced through breeding. As further illustrated with respect to the F3 and F4 generations, plants may be crossed with gene-edited plants and resulting crosses may be gene edited. It should be understood that the same could be true of plants in the F1 and/or F2 generations.

These determinations may be aided by the in silico simulations 190 of subsequent generations of the lines, which may further involve crossing the progeny with other seed lines, the introduction of particular genetics via gene editing 160, and/or the adjustment of environmental and/or crop management variables. Like predictive crossing 115, predictive recombination 175 not only looks toward future product, but may also look toward improving the genetic variability. As shown in FIG. 1A, similar predictions may be made for plants emerging from speed breeding into F3.

As further illustrated in FIG. 1A, once the seeds emerge from speed breeding into F3, decisions may be further governed by predictive advancement model 180. Predictive advancement model 180 uses the same database as the predictive crossing and predictive recombination models, but assesses the available data differently. In particular, advancement decisions made by the predictive advancement model 180 are based on expected future performance and ability of quickly achieving commercialization for each variety at least in the portion of the pipeline illustrated in association with predictive advancement 180 in FIG. 1A. As the seeds progress from F3 into F4 and beyond, the expected performance considerations considered by the system and methods shift more toward commercialization considerations/metrics.

As the plant-based product pipeline progresses, the predictive deployment model 185 is applied to make decisions about when, how, and where in the ground to plant each particular seed type in the pipeline and how to subsequently manage those plantings, including when to harvest. The predictive deployment model 185 assesses the probabilities of meeting the product specification using a particular type of seed (based on information in the seed object record 200) in a particular location, at a particular time, using particular management techniques. The predictive deployment model 185 assesses each of the potential options and ranks them. The seeds are subsequently planted for yield & increase and commercialization based on the recommendations provided by the model.

In silico simulations 190 allow the system to test alternatives that cannot be readily tested in the real world because, among other things there are just too many possibilities to test. By picking seed objects that are believed to have a higher chance of success, modeling their progeny using in silico simulations 190 and the various machine learning options, the probability of hitting the desired improved plant-based product increases. The general framework of in silico (stochastic) simulation 190 for plant breeding programs is well-known: See, e.g., Faux A M, Gorjanc G, Gaynor R C, Battagin M, Edwards S M, Wilson D L, Hearne S J, Gonen S, Hickey J M. AlphaSim: Software for Breeding Program Simulation. Plant Genome. 2016 November; 9(3). doi: 10.3835/plantgenome2016.02.0013. PMID: 27902803. (AlphaSim simulates breeding programs in a series of steps: (i) simulate haplotype sequences and pedigree; (ii) drop haplotypes into the base generation of the pedigree and select single-nucleotide polymorphism (SNP) and quantitative trait nucleotide (QTN); (iii) assign QTN effects, calculate genetic values, and simulate phenotypes; (iv) drop haplotypes into the burn-in generations; and (v) perform selection and simulate new generations.); Mackay I, Ober E, Hickey J. GplusE: beyond genomic selection. Food Energy Secur. 2015; 4(1):25-35. doi:10.1002/fes3.52 (collection of high-throughput phenotype data from field trials (HTP) (Montes et al. 2007; Araus and Cairns 2014) can be integrated directly into plant breeding programs in a manner analogous to the use of high-density marker data in genomic selection (GS) (Meuwissen et al. 2001; Jannink et al. 2010); and Liu H, Tessema B B, Jensen J, Cericola F, Andersen J R, Sorensen A C. ADAM-Plant: A Software for Stochastic Simulations of Plant Breeding From Molecular to Phenotypic Level and From Simple Selection to Complex Speed Breeding Programs. Front Plant Sci. 2019 Jan. 9; 9:1926. doi: 10.3389/fpls.2018.01926. PMID: 30687343; PMCID: PMC6333911. Each of these articles and other describe known systems and software programs that model probable outcomes when crossing and selfing particular plants.

These disclosures reference the general possibility of using particular genetic, genotypic and phenotypic data as inputs and provide particular examples for particular plants. These references can generally be extrapolated by one of ordinary skill in the art to other use cases, including soybean. However, none of them recognize the desirability of assessing the probability that a particular seed, breeding decision, or series of longitudinal breeding decisions will result in a producing a seed that meets the specification for a desired plant-based product, especially where that specification has multi-faceted requirements (e.g., higher protein, better taste, improved water holding capacity).

As further illustrated, candidate lists 115 and 120 may have elements that are based on one another. For instance, the ranked list of potential crosses 115 may include a cross involving the progeny of a gene edited plant as recommended in list 120. Conversely, the list of gene editing targets 120 may rely upon the progeny of a potential cross recommended in list 115.

Portions of ranked lists 115 and 120 are used as the basis for a selective breeding program (150) and a gene editing program (160), respectively.

### Gene Editing Enabled by Machine Learning

Genetic editing 160 is different from the transgenic, or “GMO,” approach in that it advances natural genetic variation that could be achieved using traditional breeding approaches rather than introducing genes foreign to the species, as is the case in GMO technology.

One method for gene editing that may be used to achieve this non-transgenic approach is called CRISPR. CRISPR technology is well-known. Generally speaking, the CRISPR nuclease scans the genome for the target site within the existing genome of the plant and makes a precise cut in the DNA. The DNA reattaches at the target site with the intended edit, leveraging the native genetic code.

The machine learning model predicts a probability ranked list potential gene editing candidates 120 using genotypic and phenotypic data including data regarding an orthologous species. An “ortholog” is a gene in a different species that has evolved through speciation events only.” Getting Started in Gene Orthology and Functional Analysis (2010) (https://www.ncbi.nlm.nih.gov/pmc/articles/PMC2845645/). Identifying orthologs helps to identify phenotypic information regarding genes with similar functionality. The same advantages may be seen with orthologous promoters.

As further illustrated in FIG. 1, following the F4 generation of the breeding program 150 and the results of the gene editing program 160, potentially advancing lines are at least partially (if not wholly) sequenced and the resulting genome for each potentially advancing line is analyzed by the one or more machine learning models (180) to determine the probability of whether the desired specification will be met by commercial production of that line on the farm in a field. Those lines for which the probability meets pre-determined criteria are advanced to farm field trials (190). In the farm field trials 190, phenotypic data is gathered for analysis by the ML system. Genomic data may also be gathered from certain plants during the farm field trials (190). If the data meets the pre-determined threshold(s), the plant products are advanced for ingredient processing (195). Data is collected on the processed ingredients, which is considered by the ML model(s) to determine whether or not the ingredients sufficiently meet the specifications. This may include phenotypic, genomic, and sensory panel data.

In some embodiments, the method may determine expression change for plant genes. In some embodiments, the method may use transcriptomic data (RNA-seq expression matrix) in combination with genotype data to build the machine learning Expression Predictive Model. In some embodiments, the machine learning model may employ ElasticNet implementation in Python, allowing parallelization and hyperparameter tuning across multiple parameters. In some embodiments, the method may separate gene models built for each gene, which are used to predict gene expression for one or more genes of a plant genome. In some embodiments, the method may use the predicted expression and Random Forest model to predict phenotype. In some embodiments, the method may report the predictive accuracy for predicted phenotypes. In some embodiments, the method may report feature importance and Shapley values for the contribution to each gene. In some embodiments, the method provides directionality for the effect of a given edit on the desired phenotype and ranks candidate gene edits based on the predicted effect. In some embodiments, the method may provide single or combinatorial gene targets. All of the features can be implemented using, for example, this system architecture described herein.

Thus, the machine learning models may be operated toward recommending the selection of one or more candidate genomic edits and prediction of the cumulative effect of the recommended edits on given agronomic traits. The machine learning model may determine candidate genes and directionality of expression change. In particular, the system may implement a method for determining expression change for plant genes comprising: (A) predicting gene expressions for one or more genes of a plant genome using a first machine learning model that takes as input genotype information; (B) determining functional relationships between features of the gene expressions and a plurality of phenotypes using a second machine learning model that takes as input data indicative of the gene expressions; and (c) generating data indicative of directionality for at least one of the gene expressions based on the functional relationships.

The method may use a high-throughput transcriptomic and genotype dataset to build a first machine learning model that predicts genetically regulated expression using genotype information. The method may feed expression data into a second machine learning model, which can account for non-linear dynamics and interactivity between genes, providing high global predictive accuracy. The method may employ the functional relationships between the gene expression features and phenotypes derived by the model to advise recommendations for gene editing strategy. Gene editing recommendations may comprise single editing targets, as well as multiple editing strategies, that involve balancing genes with interactive expression patterns. The method may provide directionality for how edits will affect the desired phenotype.

Data gathered at every stage may be included as part of the training data toward improving and/or even re-establishing one or more of the machine learning models for future use within the system. As seeds progress through the pipeline, the information regarding the seed and the ability to calculate its probability of successfully meeting the product specification increases. Conversely, as the seeds progress through the pipeline the number of alternative paths decreases.

### Example Applications of the Machine Learning Model

Example 1: Soy, specifically soy protein concentrate (SPC) is the number one protein ingredient used in plant-based meat applications. SPC has a protein content of approximately 65%. Currently, SPC is primarily made by processing of defatted soy flour (approximately 47% protein content) produced from soybeans with an average protein content of approximately 36%. The processing required to increase the protein content is costly, water-intensive, and energy-intensive. It is believed that an ultra-high protein soybean could make this process less expensive, less water-intensive, and more energy-efficient. By leveraging the soybean plant's genetic diversity, its protein content may be increased to a sufficiently high-level (at least 49%) that it would effectively disintermediate one or more processing steps necessary to arrive at the protein level suitable for plant-based meat applications. As the protein content of the soybeans in the field is driven toward 65%, the less waste and processing that would be required to produce Soy Protein Concentrate.

Example 2: Through machine learning it is anticipated that better soybean genetics can be found in a germplasm which includes, among other varieties, the wild ancestor of the present day commercial soybean, Glycine soja (previously G. ussuriensis) or created using lessons from that broader germplasm and/or orthologs that will (a) facilitate other easier, cheaper, more environmentally friendly production of soy-based ingredients, potentially alleviating supply constraints; (b) allow for the production of completely new ingredients (e.g., de-flavored, high-water holding capacity soybeans for enhanced flavor and texture in final plant-based meat products; healthy oils (due to higher oleic acid); stable gelation); (c) new food products; and/or (d) improved end user satisfaction (e.g., better taste, texture, color).

Example 3: Soybean meal is an ideal protein source for swine, poultry, and fish due to its availability, cost, high protein content, and balanced amino acid profile. In fact, currently over 90% of the soybeans produced in the United States are fed to animals. However, its use has been restricted because—like many plant proteins—soybean meal has a high concentration of antinutritional compounds (ANCs), including oligosaccharides such as raffinose and stachyose that can have a negative effect on protein digestibility, leading to low energy values, poor metabolism, and excessive secretion impacting water quality in aquaculture systems. Apart from antinutritional factors, the steady decline in protein content of soy—an unintended consequence of breeding primarily for yield and other agronomic traits—has rendered soy meal a continually less valuable feed ingredient. Through machine learning it is anticipated that the expression of oligosaccharides such as raffinose and stachyose can be significantly decreased.

Example 4: The yellow pea is another significant source of plant-protein. Currently, pea protein concentrate (PPC) has a protein content of approximately 50-52% and pea protein isolate (PPI) a protein content of about 85%. The flavor and color of PPC is not preferred by consumers. While PPI has better flavor, the cost of process is much higher. Using machine learning to understand and optimize the diversity of yellow pea parents in a germplasm and subsequently optimize and prioritize the crosses most likely to succeed, it is believed that the protein content of the yellow pea in the field can be significantly increased much like the protein content of unprocessed soybeans is being increased. It is also believed that machine learning models will help identify the gene(s) that result in the undesirable flavor and color of the yellow pea and provide gene editing actions to mute/lessen the undesirable flavor and color to provide greater consumer interest in yellow-pea based food ingredients. This will (a) facilitate other easier, cheaper, more environmentally friendly production of yellow-pea-based ingredients, alleviating plant-protein supply constraints; (b) allow for the production of completely new ingredients (e.g., de-flavored, high-water holding capacity yellow peas for enhanced flavor and texture in final plant-based meat products); (c) new food products; and/or (d) improved end user satisfaction (e.g., better taste, texture, color).

### Computing Environment to Support Machine Learning

It should also be noted that the machine learning models, data collection, various logic and/or functions disclosed herein may be enabled using any number of combinations of hardware, firmware, and/or as data and/or instructions embodied in various machine-readable or computer-readable media, in terms of their behavioral, register transfer, logic component, and/or other characteristics. Computer-readable media in which such formatted data and/or instructions may be embodied include, but are not limited to, non-volatile storage media in various forms (e.g., optical, magnetic or semiconductor storage media) and carrier waves that may be used to transfer such formatted data and/or instructions through wireless, optical, or wired signaling media or any combination thereof. Examples of transfers of such formatted data and/or instructions by carrier waves include, but are not limited to, transfers (uploads, downloads, e-mail, etc.) over the Internet and/or other computer networks via one or more data transfer protocols (e.g., HTTP, FTP, SMTP, and so on).

Aspects of the methods and systems described herein, such as the logic or machine learning models, may be implemented as functionality programmed into any of a variety of circuitry, including programmable logic devices (“PLDs”), such as field programmable gate arrays (“FPGAs”), programmable array logic (“PAL”) devices, electrically programmable logic and memory devices and standard cell-based devices, as well as application specific integrated circuits. Some other possibilities for implementing aspects include: memory devices, microcontrollers with memory (such as EEPROM), embedded microprocessors, firmware, software, etc. Furthermore, aspects may be embodied in microprocessors having software-based circuit emulation, discrete logic (sequential and combinatorial), custom devices, fuzzy (neural) logic, quantum devices, and hybrids of any of the above device types. The underlying device technologies may be provided in a variety of component types, e.g., metal-oxide semiconductor field-effect transistor (“MOSFET”) technologies like complementary metal-oxide semiconductor (“CMOS”), bipolar technologies like emitter-coupled logic (“ECL”), polymer technologies (e.g., silicon-conjugated polymer and metal-conjugated polymer-metal structures), mixed analog and digital, and so on.

Aspects of the methods and systems disclosed herein may be embodied and/or executed by the logic of the processes described herein, which may also be embodied in the form of software instructions and/or firmware that may be executed on any appropriate hardware. For example, logic embodied in the form of software instructions and/or firmware may be executed on a dedicated system or systems, on a personal computer system, on a distributed processing computer system, and/or the like. In some embodiments, logic may be implemented in a stand-alone environment operating on a single computer system and/or logic may be implemented in a networked environment such as a distributed system using multiple computers and/or processors, for example.

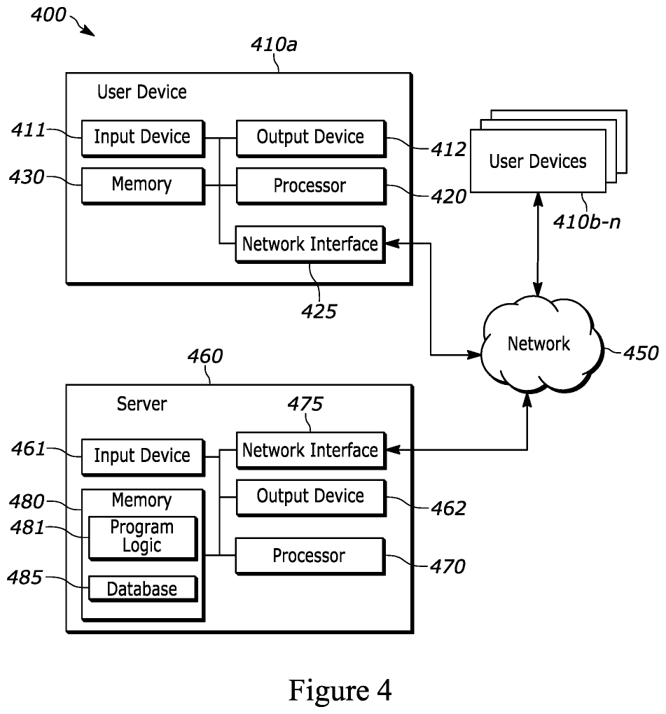

Aspects of the methods and systems described herein may also be implemented on an illustrative system 400, depicted in association with FIG. 4. In particular, system 400 may comprise a user devices 410a-n, server 460, and network 450.

The user device 410 of the system 400 may include various components including, but not limited to, one or more input devices 411, one or more output devices 412, one or more processors 420, a network interface device 425 capable of interfacing with the network 450, one or more non-transitory memories 430 storing processor executable code and/or software application(s), for example including, a web browser capable of accessing a website and/or communicating information and/or data over the network, and/or the like. The memory 430 may also store an application (not shown) that, when executed by the processor 420 causes the user device 410 to provide the functionality of the various systems and methods described the present specification, as would be understood by those of ordinary skill in the art having the present specification, drawings, and claims before them.

The input device 411 may be capable of receiving information input from the user and/or processor 420, and transmitting such information to other components of the user device 410 and/or the network 450. The input device 411 may include, but are not limited to, implementation as a keyboard, touchscreen, mouse, trackball, microphone, remote control, and combinations thereof, for example.

The output device 412 may be capable of outputting information in a form perceivable by the user and/or processor 420. For example, implementations of the output device 412 may include, but are not limited to, a computer monitor, a screen, a touchscreen, an audio speaker, a website, and combinations thereof, for example. It is to be understood that in some exemplary embodiments, the input device 411 and the output device 412 may be implemented as a single device, such as, for example, a computer touchscreen. It is to be further understood that as used herein the term “user” is not limited to a human being, and may comprise, a computer, a server, a website, a processor, a network interface, a user terminal, and combinations thereof, for example.

The server 460 of the system 400 may include various components including, but not limited to, one or more input devices 461, one or more output devices 462, one or more processors 470, a network interface device 475 capable of interfacing with the network 450, and one or more non-transitory memories 480 for storing data structures/tables (including those of database 485) that may be used by the system 400 and particularly server 460 to perform the functions and procedures set forth herein. The memory 480 may also store an application/program store 481 that, when executed by the processor 470 causes the server 460 to provide the functionality of the systems and methods disclosed in the present application.

As shown in FIG. 4, the server 460 may include a single processor or multiple processors working together or independently to execute the program logic 481 stored in the memory 480 as described herein. It is to be understood, that in certain embodiments using more than one processor 470, the processors 470 may be located remotely from one another, located in the same location, or comprising a unitary multi-core processor. The processors 470 may be capable of reading and/or executing processor executable code and/or capable of creating, manipulating, retrieving, altering, and/or storing data structures and data tables (including those of database 485) into the memory 480.

Exemplary embodiments of the processor 470 may be include, but are not limited to, a digital signal processor (DSP), a central processing unit (CPU), a field programmable gate array (FPGA), a microprocessor, a multi-core processor, combinations, thereof, and/or the like, for example. The processor 470 may be capable of communicating with the memory 480 via a path (e.g., data bus). The processor 470 may be capable of communicating with the input device 461 and/or the output device 462.

The input device 461 of the server 460 may be capable of receiving information input from the user and/or processor 470, and transmitting such information to other components of the server 460 and/or the network 450. The input device 461 may include, but are not limited to, implementation as a keyboard, touchscreen, mouse, trackball, microphone, remote control, and/or the like and combinations thereof, for example. The input device 461 may be located in the same physical location as the processor 470, or located remotely and/or partially or completely network-based.

The output device 462 of the server 460 may be capable of outputting information in a form perceivable by the user and/or processor 470. For example, implementations of the output device 462 may include, but are not limited to, a computer monitor, a screen, a touchscreen, an audio speaker, a website, a computer, and/or the like and combinations thereof, for example. The output device 462 may be located with the processor 470, or located remotely and/or partially or completely network-based.

The memory 480 stores applications or program logic 481 as well as data structures (including those of database 485) that may be used by the system 400 and particularly server 460. The memory 480 may be implemented as a conventional non-transitory memory, such as for example, random access memory (RAM), CD-ROM, a hard drive, a solid state drive, a flash drive, a memory card, a DVD-ROM, a disk, an optical drive, combinations thereof, and/or the like, for example. In some embodiments, the memory 480 may be located in the same physical location as the server 460, and/or one or more memory 480 may be located remotely from the server 460. For example, the memory 480 may be located remotely from the server 460 and communicate with the processor 470 via the network 450. Additionally, when more than one memory 480 is used, a first memory 480a may be located in the same physical location as the processor 470, and additional memory 480n may be located in a location physically remote from the processor 470. Additionally, the memory 480 may be implemented as a “cloud” non-transitory computer readable storage memory (i.e., one or more memory 480 may be partially or completely based on or accessed using the network 450).

Each element of the server 460 may be partially or completely network-based or cloud-based, and may or may not be located in a single physical location. As used herein, the terms “network-based,” “cloud-based,” and any variations thereof, are intended to include the provision of configurable computational resources on demand via interfacing with a computer and/or computer network, with software and/or data at least partially located on a computer and/or computer network. In other words, the server 460 may or may not be located in single physical location. Additionally, multiple servers 460 may or may not necessarily be located in a single physical location.

Database 485 may comprise one or more data structures and/or data tables stored on non-transitory computer readable storage memory 480 accessible by the processor 470 of the server 460. The database 485 can be a relational database or a non-relational database. Examples of such databases include, but are not limited to: DB2®, Microsoft® Access, Microsoft® SQL Server, Oracle®, mySQL, PostgreSQL, MongoDB, Apache Cassandra, and the like. It should be understood that these examples have been provided for the purposes of illustration only and should not be construed as limiting the presently disclosed inventive concepts. The database 485 can be centralized or distributed across multiple systems.

The teachings herein are not limited to certain plant species, and it is envisioned that they can be modified to be useful for monocots, dicots, and/or substantially any crop and/or valuable plant type, including plants that can reproduce by self-fertilization and/or cross fertilization, hybrids, inbreds, varieties, and/or cultivars thereof. Some of example plant species include, soybeans (Glycine max), peas (Pisum sativum and other members of the Fabaceae like Cjanus and Vigna species), chickpeas (Cicer arietinum), peanuts (Arachis hypogaea), lentils (Lens culinaris or Lens esculenta), lupins (various Lupinus species), mesquite (various Proopis species), clover (various Trifolium species), carob (Ceraionia siliqua), tamarind, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), camelina (Camelina sativa), millet (e.g., pearl millet (Pennisetum glaucum), proso millet (Panicum miliaceum), foxtail millet (Setaria italica), finger millet (Eleusine coracana)), sunflower (Helianthus annuus), quinoa (Chenopodium quinoa), chicory (Cichorium intybus), tomato (Solanum lycopersicum), lettuce (Lactuca sativa), safflower (Carthamus tinctorius), wheat (Triticum aestivum), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oil palm (Elaeis guineensis), poplar (Populus spp.), eucalyptus (Eucalyptus spp.), oats (Avena sativa), barley (Hordeum vulgare), flax (Linum usitatissimum), Buckwheat (Fagopyrum esculentum) vegetables, ornamentals, and conifers.

While particular embodiments of the present invention have been shown and described, it should be noted that changes and modifications may be made without departing from the presently disclosed inventive concepts in its broader aspects and, therefore, the aim in the appended claims is to cover all such changes and modifications as fall within the true spirit and scope of this invention.

What is claimed is:

1. A method for training a machine-learning model and subsequently applying that machine learning model to accelerate speed to market for an improved plant-based product, one or more seeds contributing to the improvement of the plant-based product comprising:
collecting into a database, with a processor, seed data for a plurality of seed varieties within a germplasm, such seed data comprising at least labelled parentage information that includes genetics information;
training, with the processor, a first machine learning model based on the data collected for each data type for each of the plurality of seed varieties within the germplasm;
establishing, via the processor, a functional specification for the improved plant-based product;
extracting, with the processor, one or more plant traits needed to at least meet the functional specification;
inputting, via the processor, the one or more plant traits needed to at least meet the functional specification into the trained first machine learning model to generate a first predictive breeding crosses list ranked based on aggregate probability that a progeny of the cross will substantially conform to one or more of the one or more plant traits needed to meet the functional specification and a first list of potential gene editing targets based on a probability that editing a particular gene will result in a plant that will substantially conform to one or more of the one or more plant traits needed to at least meet the functional specification;
collecting data, by the processor, from the progeny of crosses planted based on the first predictive breeding crosses list; and
comparing, by the processor, the collected progeny data to corresponding predictions made by the first machine learning model toward determining next action recommended by the first machine learning model.

2. The method according to claim 1 further comprising:
selecting, with the processor, a second machine learning model based on the data type of each data element of the training data selected to train the second machine learning model (“second training data”), the second machine learning model selected from the group comprising supervised learning models, unsupervised learning models, and combinations thereof and different from the first machine learning model;
training, with the processor, the second machine learning model using the second training data from the database;
inputting, via the processor, the one or more plant traits needed to at least meet the functional specification into the trained second machine learning model to generate a second predictive breeding crosses list ranked based on aggregate probability that a progeny of the cross will substantially conform to one or more of the one or more plant traits needed to meet the functional specification and a second list of potential gene editing targets based on a probability that editing a particular gene will result in a plant that will substantially conform to one or more of the one or more plant traits needed to at least meet the functional specification;
collecting data, by the processor, from the progeny of crosses planted based on the second predictive breeding crosses list; and
comparing the collected progeny data to corresponding predictions made by the second machine learning model toward determining next action recommended by the second machine learning model.

3. The method according to claim 2 further comprising
mediating between the first machine learning model and the second machine learning model to establish an aggregated predictive breeding crosses list based on the first and second predictive breeding crosses lists;
collecting data from the progeny of crosses planted based on the aggregated predictive breeding crosses list; and
comparing the collected progeny data to corresponding predictions made by both the first and the second machine learning models toward determining next action recommended by the first and second machine learning model; and
mediating between the first machine learning model and the second machine learning model to determine the best next action recommendation.

4. The method according to claim 3 wherein the first machine learning model is paired with an in silico simulation model.

5. The method according to claim 1 wherein the first machine learning model is paired with an in silico simulation model.
